(19) United States
(12) Patent Application Publication
(10) Pub. No.: US 2008/0171980 A1
(43) Pub. Date: Jul. 17, 2008

(54) Proximal Shaft for Rapid Exchange Catheter

(75) Inventor: Luke Hughes, Co. Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/623,351

(22) Filed: Jan. 16, 2007

(51) Int. Cl. A61M 25/10

(52) U.S. Cl. 604/103.04

(57) ABSTRACT

A rapid exchange catheter is disclosed having a single lumen proximal shaft portion with a helical coil or helically cut hypotube component extending within a lumen thereof. The helical coil or helically cut hypotube component provides stiffness and pushability to the catheter. A proximal end of helical coil or helically cut hypotube component is attached within a luer fitting at a proximal end of the proximal shaft portion, such that the helical coil or helically cut hypotube component extends within the proximal shaft lumen in a somewhat cantilevered arrangement unsupported and unattached at any point along its length with the proximal shaft portion. As such, the helical coil or helically cut hypotube component moves independent of the proximal shaft portion to adjust to the twists and turns of the vasculature independent of the proximal shaft portion.

### FIELD OF THE INVENTION

The invention relates to monorail or rapid exchange dilatation catheters, and more particularly to a proximal shaft construction of utility in this type of catheter.

### BACKGROUND OF THE INVENTION

Monorail or rapid exchange dilatation catheters are preferred by some clinicians for angioplasty procedures because, unlike over-the-wire catheters that require guidewire extenders to perform a catheter exchange, the monorail dilatation catheter construction enables one monorail dilatation catheter to be exchanged readily for another. Monorail dilatation catheters generally have a guidewire lumen only in a distal segment of the catheter, through which the catheter and a guidewire may be coupled together. Conventionally, a guidewire tube or shaft extends through a dilatation balloon from a distal end of the catheter to a point proximal to the balloon. Rapid exchange and manipulation of the dilatation catheter is facilitated because the catheter segment in contact with the surface of the guidewire is only as long as the balloon.

Although conventional monorail-type catheters allow rapid catheter exchange, they tend to lack stiffness along their shaft length proximal to a proximal guidewire port, at which point, the catheter gains the benefit of the guidewire to impart stiffness to the distal portion of the catheter. A lack of stiffness along the proximal portion of the catheter may make it difficult to advance the catheter through a body lumen or cause the unsupported proximal portion of the catheter shaft to buckle.

To provide a proximal shaft of a monorail catheter with sufficient pushability and crossability, while maintaining trackability within the patient's tortuous vasculature, prior art designs have supplemented polymer catheter shafts with a support mandrel or stiffening wire. Often a separate lumen is provided for the mandrel, adding to the profile of the catheter, or the mandrel or wire is attached to or secured within the walls of the proximal shaft. However, such mandrels or stiffening wires may still buckle under compressive loads and present a potential risk of perforating the catheter wall.

Other conventional proximal shaft designs have addressed handling and performance issues by using materials of different stiffness for the proximal and distal portions of the catheter and employing a high strength metallic proximal shaft section, commonly called a hypotube. Issues that arise with such arrangements include kinking at the junction of the proximal and distal shaft portions that then necessitates the use of reinforcing layers, stiffening wires or other measures to ease the transition in stiffness between the catheter shaft materials. Complicated bonds between the different materials of the proximal and distal shaft portions are also a drawback to the use of a hypotube as the proximal shaft component.

As such, a need still exists for a rapid exchange dilatation catheter having a proximal shaft component that is flexible but that possesses the requisite pushability for performing within a patient's vasculature without buckling and/or perforating the proximal catheter shaft.

### BRIEF SUMMARY OF THE INVENTION

The present invention is directed toward a proximal shaft component for a rapid exchange dilatation catheter. An embodiment includes a catheter having an elongate catheter shaft with an inflation lumen extending from a proximal end to a distal end thereof. The catheter shaft has a single lumen proximal shaft portion that extends from the proximal end of the catheter shaft to a proximal guidewire port and a dual lumen distal shaft portion that extends from the proximal guidewire port to the distal end of the catheter shaft. A luer fitting is attached to the proximal end of the catheter shaft for delivering an inflation fluid to the inflation lumen for delivery to a dilatation balloon attached to the distal end of the catheter shaft. A helical coil component freely extends within the inflation lumen of the proximal shaft portion and includes a proximal end attached within the luer fitting, a distal end positioned adjacent to the proximal guidewire port and an outer diameter that is less than a diameter of the inflation lumen of the proximal shaft portion.

In another embodiment, a hypotube component having a helical cut from a proximal end to a distal end thereof is positioned within the inflation lumen of the proximal shaft component. The proximal end of the helically cut hypotube component is attached within the hub and the distal end is positioned adjacent to the proximal guidewire port, such that the hypotube component is unattached and unsecured along its length that extends within the proximal shaft portion. The hypotube component has an outer diameter that is less than a diameter of the inflation lumen of the proximal shaft portion, such that the helically cut hypotube component moves independent of the proximal shaft portion within the inflation lumen thereof.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a side view in partial cross-section of a rapid exchange catheter according to an embodiment of the present invention.

FIG. 2 is a sectional view of an area adjacent to proximal guidewire port 112 of the catheter shown in FIG. 1.

FIG. 3 is a sectional view of an area adjacent to proximal guidewire port 112 of the catheter shown in FIG. 1 according to another embodiment of the present invention.

FIG. 4 illustrates a side view in partial cross-section of a rapid exchange catheter according to another embodiment of the present invention.

FIG. 5 illustrates a side view of a hypotube component according to an embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

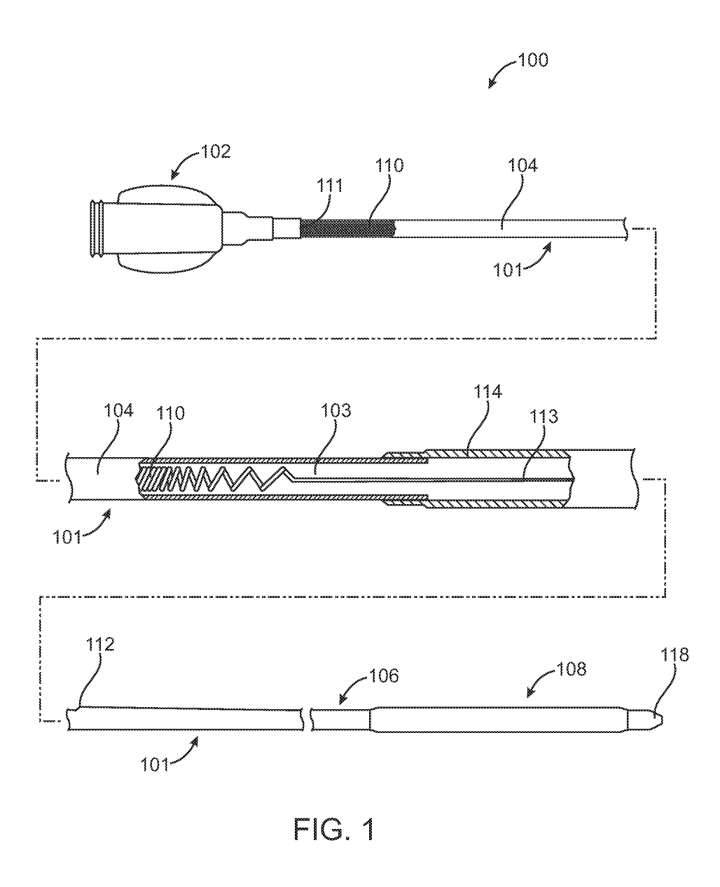

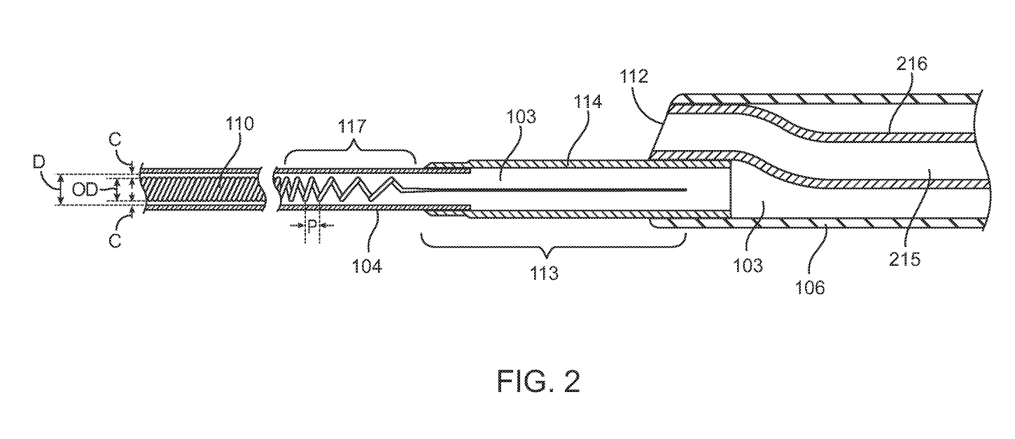

FIGS. 1 and 2 illustrate a rapid exchange dilatation catheter 100 according to an embodiment of the present invention. Catheter 100 has an elongate catheter shaft 101 having an inflation lumen 103 extending from a proximal end to a distal end thereof. A luer fitting or hub 102 is attached to the proximal end of catheter shaft 101 for delivering an inflation fluid to inflation lumen 103. A dilatation balloon 108 is attached to the distal end of catheter shaft 101 in fluid communication with inflation lumen 103 for receiving the inflation fluid.

Dilatation balloon 108 may be made from a thermoplastic material or a thermoplastic elastomer, such as various polyesters and their block co-polymers, polyamides and their block co-polymers, and polyurethane block co-polymers. Dilatation balloon 108 according to various embodiments of the present invention may be made from materials and by methods disclosed in the following patents and patent publication: U.S. Pat. No. 5,290,306 to Trotta et al., U.S. Pat. No. 6,171,278 to Wang et al., U.S. Pat. Nos. 6,210,364 B1, 6,283,939 B1 and 5,500,180, all to Anderson et al., and U.S. patent application Publ. No. 2006/0134357 to Godaire et al., each of which is incorporated by reference herein in its entirety.

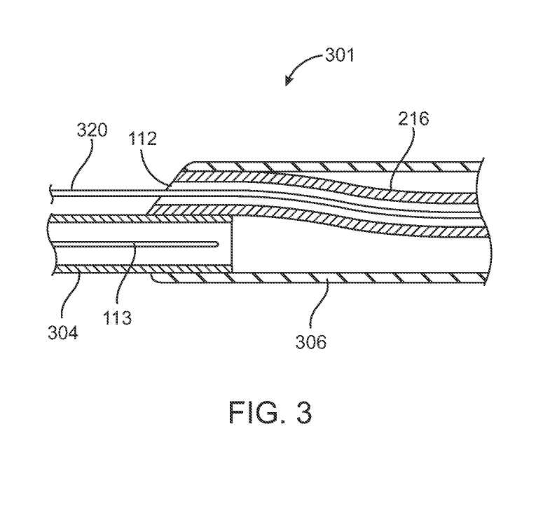

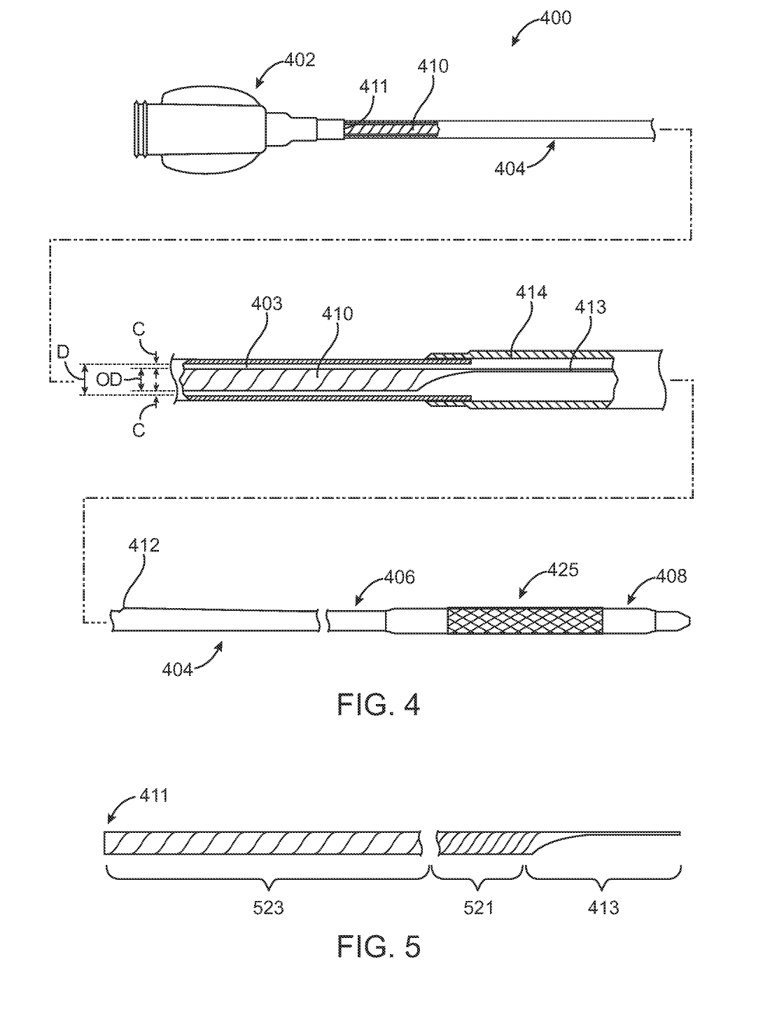

In use in an angioplasty procedure, dilatation catheter 100 is tracked through the vascular system of a patient until balloon 108 is located within a stenosis at a treatment site of a vessel. Once positioned, balloon 108 of catheter 100 is inflated by introduction of an inflation fluid through luer fitting 102 to bear against the stenosis and thereby enlarge the opening of the vessel. In a further embodiment shown in FIG. 4, a stent delivery catheter 400 according to the present invention may include a dilatation balloon 408 with an expandable stent 425 positioned thereon for deployment either following an angioplasty treatment or during initial balloon dilatation of the treatment site, which is referred to as primary stenting. Catheters 100, 400 are typically guided to the treatment site by a guidewire, such as guidewire 320 shown in FIG. 3.

Catheter shaft 101 includes a single lumen proximal shaft portion 104 that extends from the proximal end of catheter shaft 101 to a proximal guidewire port 112. Catheter shaft 101 further includes a dual lumen distal shaft portion 106 that extends from proximal guidewire port 112 to the distal end of catheter shaft 101. A guidewire tube or distal inner shaft 216 is positioned within distal shaft portion 106 and balloon 108 to provide a relatively short guidewire lumen 215 between proximal guidewire port 112 and a distal tip 118 of catheter 100. Transition tubing 114 is utilized to ease a transition in stiffness between and to facilitate bonding of proximal and distal shaft portions 104, 106. In an alternate embodiment illustrated in FIG. 3, proximal and distal shaft portions 304, 306 are made of the same polymer or readily bondable polymers such that transition tubing is not utilized, thereby simplifying the construction of catheter shaft 301.

In embodiments according to the present invention, proximal and distal shaft portions 104, 106 and distal inner shaft 216 may be made of polyethylene, PEBAX, nylon, polyurethane, or a co-extrusion or copolymer of these materials. In one embodiment, proximal shaft portion 104 is made of polyethylene and distal shaft portion 106 is made of an inner layer of polyethylene and an outer layer of PEBAX to facilitate bonding of distal shaft portion 106 to proximal shaft portion 104. In another embodiment, distal inner shaft 216 is made of an inner layer of PEBAX and an outer layer of polyethylene to facilitate bonding of distal inner shaft 216 with proximal and distal shaft portions 104, 106 within inflation lumen 103 proximate proximal guidewire port 112.

In accordance with an embodiment of the present invention illustrated in FIGS. 1-3, a helical coil or spring 110 provides stiffness and pushability to proximal shaft portion 104. A proximal end 111 of helical coil 110 is attached within luer fitting 102 and a distal end having a straight segment 113 is positioned adjacent to proximal guidewire port 112 within transition tubing 114 of catheter shaft 101. Helical coil 110 extends within inflation lumen 103 of proximal shaft portion 104 in a somewhat cantilevered arrangement, as it is not supported by or attached at any point along its length with proximal shaft portion 104. In an embodiment, an outer diameter ‘OD’ of helical coil 110 is less than a diameter ‘D’ of inflation lumen 103 within proximal shaft portion 104. In another embodiment, helical coil 110 is radially moveable along its length within inflation lumen 103 of proximal shaft portion 104. In another embodiment, a radial clearance ‘C’ exists between helical coil 110 and a wall of inflation lumen 103 of proximal shaft portion 104. In each of these arrangements, helical coil 110 moves independent of proximal shaft 104. Independent movement of helical coil 110 provides a requisite stiffness to proximal shaft portion 104 for better pushability, while avoiding the damage that is often caused in conventional proximal shaft components, i.e., where a proximal outer shaft has an attached reinforcing member along a point or points of its length that detaches from the outer shaft during an interventional procedure thereby splitting or tearing the outer shaft and compromising the inflation lumen. Radial and twisting movement of helical coil 110 within inflation lumen 103 allows the coil to adjust to the twists and turns of the vasculature independent of proximal shaft portion 104 without stressing, pulling or otherwise damaging the proximal shaft portion.

In an embodiment, a pitch ‘P’ between windings in a distal portion 117 of helical coil 110 is greater than a pitch between windings in a proximal portion of the helical coil to provide a transition in flexibility. In such an embodiment, the windings in the proximal portion of the helical coil may be tightly wound so as to minimize the pitch between adjacent windings to thereby provide the requisite stiffness to proximal shaft portion 104. In a further embodiment, the pitch between windings in distal portion 117 may gradually increase as helical coil 110 extends distally to provide a more gradual transition in flexibility. In the embodiments shown, a distalmost end of helical coil 110 includes a straight or unwound segment 113 that may further ease the transition in stiffness between proximal and distal shafts 104, 106 while still providing some reinforcement proximate proximal guidewire port 112. In an alternate embodiment, straight segment 113 may be a straight wire or ribbon that has been attached to a distal end of helical coil 110.

Helical coil 110 may be made of a stainless steel alloy, nitinol or other biocompatible metallic material. In embodiments of the present invention, helical coil 110 may be a helical extension or compression spring having windings with a circular cross-section, or may be formed from a metallic round wire or flat ribbon that has been wound into a coiled configuration.

In accordance with another embodiment of the present invention illustrated in FIGS. 4 and 5, a stent delivery catheter 400 includes a proximal shaft portion 404 having a helically or spirally cut hypotube component 410 extending within an inflation lumen 403 thereof, which provides stiffness and pushability to proximal shaft portion 404. Hypotube component 410 is a thin-walled, tubular structure of a metallic material, such as stainless steel, nitinol, or a cobalt-chromium super alloy. Hypotube component 410 includes a helical cut from a proximal end 411, wherein hypotube component 410 is attached within a hub 402, to proximate a distal end, wherein hypotube component 410 includes a skived, straight segment 413 that is positioned adjacent to a proximal guidewire port 412 within a transition tubing 414. Transition tubing 414 eases a transition in flexibility between proximal and distal shaft components 404, 406 and aids in bonding the proximal and distal shaft components 404, 406 with a distal inner shaft or guidewire tube (not shown). In an alternate embodiment as shown in FIG. 3, proximal and distal shaft portions 404, 406 may be made of the same polymer or readily bondable polymers such that transition tubing is not utilized, thereby simplifying the construction of the catheter shaft.

Similar to the embodiment of FIG. 1, helically cut hypotube component 410 extends within inflation lumen 403 of proximal shaft portion 404 in a somewhat cantilevered arrangement, as it is not supported by or attached at any point along its length with proximal shaft portion 404. In an embodiment, an outer diameter ‘OD’ of helically cut hypotube component 410 is less than a diameter ‘D’ of inflation lumen 403 within proximal shaft portion 404. In another embodiment, helically cut hypotube component 410 is radially moveable along its length within inflation lumen 403 of proximal shaft portion 404. In another embodiment, a radial clearance ‘C’ exists between helically cut hypotube component 410 and a wall of inflation lumen 403 of proximal shaft portion 404. In each of these embodiments, helically cut hypotube component 410 moves independent of proximal shaft 404. Independent movement of helically cut hypotube component 410 provides stiffness to proximal shaft portion 404 for better pushability, while avoiding the damage that is often caused in conventional proximal shaft components. Radial and twisting movement of helically cut hypotube component 410 within inflation lumen 403 allows the hypotube component to adjust to the twists and turns of the vasculature independent of proximal shaft portion 404 without stressing, pulling or otherwise damaging the proximal shaft portion.

As illustrated in FIG. 5, the helical cut within hypotube component 404 is longitudinally spaced farther apart in a proximal segment 523 of the hypotube component, such that the hypotube component is flexible yet retains sufficient stiffness to provide pushability to proximal shaft portion 404. In a transition segment 521 of hypotube component 404, wherein the flexibility of the hypotube component is preferably increased, the helical cut is longitudinally spaced closer together to ease the transition between proximal shaft portion 404 and distal shaft portion 406. In various embodiments, the flexibility of hypotube component 404 in proximal and transition segments 523, 521 may be increased, decreased, and/or varied through manipulation of the longitudinal spacing of the helical cut.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A rapid exchange dilatation catheter comprising:
an elongate catheter shaft having an inflation lumen extending from a proximal end to a distal end thereof, wherein the catheter shaft has a single lumen proximal shaft portion that extends from the proximal end of the catheter shaft to a proximal guidewire port and a dual lumen distal shaft portion that extends from the proximal guidewire port to the distal end of the catheter shaft;
a luer fitting attached to the proximal end of the catheter shaft for delivering an inflation fluid to the inflation lumen;
a dilatation balloon attached to the distal end of the catheter shaft in fluid communication with the inflation lumen for receiving the inflation fluid; and
a helical coil having a proximal end attached within the luer fitting, a distal end positioned adjacent to the proximal guidewire port and an outer diameter that is less than a diameter of the inflation lumen of the proximal shaft portion, wherein the helical coil freely extends within the inflation lumen of the proximal shaft portion.

2. The catheter of claim 1, wherein the helical coil is radially moveable within the inflation lumen of the proximal shaft portion.

3. The catheter of claim 1, wherein the helical coil is unsecured within the inflation lumen of the proximal shaft portion.

4. The catheter of claim 1, wherein a radial clearance exists between the helical coil and a wall of the inflation lumen of the proximal shaft portion.

5. The catheter of claim 1, wherein a pitch between windings in a distal portion of the helical coil is greater than a pitch between windings in a proximal portion of the helical coil.

6. The catheter of claim 5, wherein the pitch between windings in the distal portion increases in a distal direction.

7. The catheter of claim 6, wherein the distal most end of the helical coil includes a straight segment.

8. The catheter of claim 1, further comprising:
a guidewire tube positioned within the distal shaft portion and the balloon that extends between the proximal guidewire port and a distal tip of the catheter.

9. A rapid exchange catheter comprising:
an elongate catheter shaft having an inflation lumen extending from a proximal end to a distal end thereof, wherein the catheter shaft has a single lumen proximal shaft portion that extends from the proximal end of the catheter shaft to a proximal guidewire port and a dual lumen distal shaft portion that extends from the proximal guidewire port to the distal end of the catheter shaft;
a hub attached to the proximal end of the catheter shaft for delivering an inflation fluid to the inflation lumen;
a balloon attached to the distal end of the catheter shaft in fluid communication with the inflation lumen for receiving the inflation fluid; and
a hypotube component having a helical cut from a proximal end to a distal end thereof, the proximal end being attached within the hub and the distal end being positioned adjacent to the proximal guidewire port and having an outer diameter that is less than a diameter of the inflation lumen of the proximal shaft portion, wherein the helically cut hypotube component freely extends within the inflation lumen of the proximal shaft portion.

10. The catheter of claim 9, wherein the hypotube component is radially moveable within the inflation lumen of the proximal shaft portion.

11. The catheter of claim 9, wherein the hypotube component is unsecured within the inflation lumen of the proximal shaft portion.

12. The catheter of claim 9, wherein a radial clearance exists between the hypotube component and a wall of the inflation lumen of the proximal shaft portion.

13. The catheter of claim 9, wherein the helical cut in a distal portion of the hypotube component is closer together than the helical cut in a proximal portion of the hypotube component.

14. The catheter of claim 13, wherein a distance between the helical cut in the distal portion decreases in a distal direction.

15. The catheter of claim 14, wherein the distalmost end of the hypotube component includes a skived segment.

16. The catheter of claim 9, further comprising:
a guidewire tube positioned within the distal shaft portion and the balloon that extends between the proximal guidewire port and a distal tip of the catheter
